(19) United States
(12) Patent Application Publication
(10) Pub. No.: US 2021/0401564 A1
(43) Pub. Date: Dec. 30, 2021

(54) METHODS AND SYSTEMS FOR IMPLANTABLE MEDICAL DEVICES AND VASCULARIZATION MEMBRANES

(71) Applicants: Arizona Board Regents on Behalf of the University of Arizona, Tucson, AZ (US); PROCYON TECHNOLOGIES LLC, Tucson, AZ (US)

(72) Inventors: Steven Kent NEUENFELDT, Tuscon, AZ (US); Robert C. JOHNSON, Tuscon, AZ (US)

(21) Appl. No.: 17/279,474

(22) PCT Filed: Sep. 24, 2019
(86) PCT No.: PCT/US19/52728

§ 371 Date: Mar. 24, 2021

Related U.S. Application Data
(60) Provisional application No. 62/735,697, filed on Sep. 24, 2018.
(60) Provisional application No. 62/736,244, filed on Sep. 25, 2018.

(51) Int. Cl. A61F 2/02; A61L 27/38; A61L 27/56; A61L 27/16

(52) U.S. Cl. CPC A61F 2/022; A61L 27/3886; A61L 27/16; A61L 27/3804; A61L 27/56; A61L 27/3834

(57) ABSTRACT

An implantable medical device and methods for making and using the same are provided. In various embodiments, the device comprises a central hub structure in communication with at least one housing or pod capable of containing cells and therapeutic materials. Also provided are membrane structures and methods of forming the same, the membranes comprising a gradient of varying porosity for use with devices of the present disclosure, as well as other uses.

This International Application claims the benefit of priority of U.S. Provisional Patent Application Ser. No. 62/735,697, filed Sep. 24, 2018, and U.S. Provisional Patent Application Ser. No. 62/736,244, filed Sep. 25, 2018, the entire disclosures of which are hereby incorporated by reference.

### FIELD

Embodiments of the present disclosure relate to the field of membrane coatings for implantable medical devices, implantable medical devices having at least one surface coated with a membrane, and methods for inhibiting fibrotic capsule formation and the formation of vascular structures at a medical implant device site. Embodiments of the present disclosure also relate to implantable devices that provide enhanced vascularization with a host, and immune-isolated devices which provide for encapsulation of live cells.

### BACKGROUND

Immuno-isolation devices designed for delivering a cellular medical therapy featuring an outer vascularizing membrane and an inner allogenic cell protective membrane are manufactured with relatively difficult and labor-intensive processes. The outer vascularizing membrane generally has a three-dimensional structure that is sufficiently open to allow cells to penetrate the membrane material. This is usually laminated or otherwise affixed to an inner immune-isolation membrane that has pores that are sufficiently large to allow biological macromolecules to freely diffuse across the membrane but prevent cells of the recipient from crossing the membrane. These membranes are typically manufactured separately, laminated together, and then affixed to an implantable medical device as part of an assembly process. The separate step by which the membranes are joined together is time consuming and difficult, and renders the membrane subject to pealing, delamination and decomposition. When such pealing or delamination occurs, the tissue surrounding the implant can react to the implanted medical device by creating local regions of fibrosis. If the implantable device contains living cells that produce a therapeutic product, the local fibrosis can lead to an environment that results in impairment of function of the encapsulated cells and possibly death of those cells. Therefore, a means of creating an outer vascularizing membrane in combination with an inner, denser immune-isolation layer that cannot delaminate or peal apart from the device would allow the development of a more stable and predictable implant with better function.

The implantable medical device field remains in need of coatings and/or membranes that overcome these and other limitations associated with multi-layer, laminated, membrane constructs.

The number of patients suffering from Type I and Type II diabetes is estimated to affect about 4.6% of the world's population. Pancreas transplantation and islet transplantation are known methods for treating diabetes. However, pancreas and islet transplantation into diabetic patients is limited to a small percent of patients who might benefit from either procedure due to the lack of available human pancreata or pancreatic islets. With the recent development of insulin secreting cells derived from human stem cells, there is a possibility of treating patients with insulin dependent diabetes through transplantation. However, such cells would be subject to rejection by the immune system of the recipient patient unless immunosuppressive drugs were administered to the patient for the rest of their life. Alternatively, insulin secreting cells could be provided with an immuno-isolating implantable device and placed in the diabetic patient to act as an insulin delivery source.

Accordingly, studies for improving the viability of islet cells and islet progenitor cells in a ported immune-isolated implantable device are being conducted.

Since the islet transplantation protocol was established, clinical islet transplantation has been regarded as a treatment method for treating type 1 diabetics. However, the low engraftment success of transplanted islet cells remains a major cause of failure of long-term blood sugar regulation. Upon implantation, it is necessary for islet cells to be successfully engrafted through revascularization and blood flow regulation within a few days after transplantation. However, transplanted islet cells are exposed to a state with low vascular density and insufficient oxygen conditions, making it difficult to achieve normal engraftment of islet cells and the ability to achieve regulated insulin secretion in the patient.

Currently, there are limited means and materials to effectively implement live cell containing immuno-isolation devices in vivo. Limitations associated with supply of adequate oxygen levels to encapsulated cells, sufficient nutrient levels to the encapsulated cells, insufficient vascularization of the implanted device and immune response to the implant, remain barriers to use of cell-containing implantable devices.

### SUMMARY

Embodiments of the present disclosure provide a single layer gradient membrane, such as a non-naturally occurring single layer polymeric or similar material gradient membrane, wherein the single layer gradient membrane comprises a gradually transitioning gradient of material density and pore sizes in the micron size range. The single layer gradient membrane is characterized by continuously variable and differing pore sizes throughout the thickness of the single layer gradient membrane (FIG. 1a).

As used herein, the terms “gradient” and “gradient membrane” relate to a polymeric or similar material membrane having an internal structure comprising gradually changing pore sizes. The pore sizes of the gradually changing pore sizes of the gradient membrane are in the micron size range. As used herein, the term “micron” is used in the singular and plural to refer to micrometer and/or micrometers.

Single component membranes of the present disclosure (i.e., single layer membranes with a non-laminated structure) are characterized by a continuous gradient of gradually transitioning pore size, from a tight or dense intertwined structure region (having relatively small pore size) to a more open or loose intertwined fiber network (having a relatively larger pore size). Progression from the inner structure/surface to the outer structure/surface of the membrane evidences a transition of gradient to a more open structural configuration. Likewise, the pores gradually transition from smaller to larger, such as from about 0.1 to about 1.0 micron at one surface (such as an inside surface), towards the outer surface of the membrane, having a membrane region comprising a gradient of pore size from about 2.0 to approximately 100 micron (or in some embodiments, from about 5 to about 15 micron) through the single layer, component membrane.

One of ordinary skill in the art will readily understand the term “pore size” as used herein. Additionally, one of ordinary skill in the art will understand and recognize different methods and devices for measuring and evaluating pore sizes. In some embodiments, pore sizes of embodiments of the present disclosure are evaluated, measured, and/or confirmed by the use of a bubble point test method or a scanning electron microscope. Single layer gradient membranes of the present disclosure comprise various materials, including those deemed appropriate by a person skilled in the art for an implantable medical device. For example, membranes of the present disclosure are contemplated as being prepared from a polymeric material. In such embodiments, the single layer gradient membrane is prepared from such polymeric materials as: polysulfone, polyarylethersulfone (PAES), polyethersulfone (PES), cellulose ester (cellulose acetate, cellulose triacetate, cellulose nitrate), nanocellulose, regenerated cellulose (RC), silicone, polyamide (nylon), polyimide, polyamide imide, polyamide urea, polycarbonate, ceramic, titanium oxide, aluminum oxide, silicon, zeolite (alumosilicate), polyarylonitrile (PAN), polyethylene (PE), low density polyethylene (LDPE), polypropylene (PP), polytetrafluoroethylene (PTFE), polyvinylidene fluoride (PVDF), polyvinylchloride (PVC), polypiperazine amide, polyethylene terephthalate (PET), polycarbonate (PC), polyurethane, and any complex or mixtures thereof. In particular embodiments, a single layer gradient membrane comprises of a polymeric material comprising polytetrafluoroethylene (PTFE). In certain preferred embodiments, PTFE is provided for at least a vascularizing layer of devices of the present disclosure. Additional materials are contemplated as being provided in membranes and implants of the present disclosure in addition to or in lieu of PTFE.

In some embodiments, a gradient membrane comprises an electro-spun polymeric membrane, such as an electrospun PTFE membrane that is applied directly to a surface, such as a surface of an implantable medical device. Implantable medical devices of the present disclosure are contemplated as comprising an internal chamber of live cells. No separate assembly steps are required to provide a protective layer/film to an internal chamber of an implantable medical device in which live cells may be contained, as the single layer gradient membrane is capable of protecting the cells from immune attack, while simultaneously permitting nutrient flow/oxygen to contained live cells, owing to the appropriate gradient pore size provided by the single layer gradient membrane. Single layer gradient membranes of the present disclosure also provide for a slightly larger pore size within the membrane region extending to the other surface (e.g., outer surface) of the single layer membrane, thus providing a surface suitable for vascularizing the outer surface of the implantable medical device in a host.

In various embodiments, single layer, gradient membranes are formed with phase inversion, interfacial polymerization, solution coating and/or phase deposition methods. These and other processes are described in Baker (Baker, R. Membrane Technology and applications. John Wiley & Sons, 2004), which is hereby incorporated by reference in its entirety.

In various embodiments, electrospinning is provided as a process to control fabricating a fibrous mat of changing and defined density in a single layer membrane construction.

It is an aspect of the present disclosure to provide materials and processes that provide for the elimination of delamination problems of prior fabricated techniques having a bi-layer membrane structure. In addition, the method by which the single layer, gradient membranes are prepared are preferable to other 2-step processes, that require a separate lamination and/or fusing step between two separately fabricated membranes, such as that described in U.S. Pat. No. 6,060,640, which is hereby incorporated by reference in its entirety. In various embodiments, implantable medical devices are provided that comprise at least one surface upon which a single layer membrane material having a gradient structure is applied. The surface is contemplated as comprising the surface of an implantable medical device, such as an implantable device that has a lumen comprising living cells (e.g. stem cells). The gradient pore size of the single layer membrane permits the passage of desired molecules, such as nutrients in an in vivo environment, to move through the membrane and to encapsulated living cells in the lumen of an implantable medical device. The single layer gradient membrane also permits passage of molecules out of the lumen of an implantable medical device, such as a therapeutic product/agent that is contained in the lumen of the implantable medical device. In this manner, the gradient single layer membrane permits the implantable medical device to act in releasing therapeutic product/agents out of the implantable medical device and available for absorption in the patient.

In various embodiments, membranes are employed as coatings on any or all surfaces of an implantable medical device. Some surfaces of an implant device may be devoid of a membrane. For example, surfaces at which fibrotic mass formation is not a significant occurrence are contemplated as being devoid of membranes. Additional surfaces that are devoid of a membrane include, for example, surfaces at a sonic weld joint on an access port of an implantable medical device.

In one embodiment, a single layer gradient membrane to reduce overall fibrosis comprises pores having a size of about 0.1 to about 100 micron (or, from about 0.1 or about 5 micron to about 15 micron). In some embodiments, an implantable medical is provided that comprises a lumen comprising living cells. The single layer gradient membrane comprises a pore size that does not interfere with the passage of molecules (such as insulin produced by contained islet cells) out of a lumen chamber (having its own chamber lining), and out of the implantable medical device into the body. In this regard, the membrane is sufficiently thin so as to allow rapid diffusion of molecules out of the implantable medical device. As another example, a single layer gradient membrane is provided on some surfaces of a component of a multi-component implantable medical device and not on other surfaces.

In certain embodiments, implant systems are provided that comprise a surface having a single layer gradient membrane, such as a membrane comprising a polymeric material. By way of example, the polymeric material is contemplated as comprising PTFE, where the PTFE membrane comprises a gradient of pore sizes. This single layer PTFE gradient membrane is provided to the external surface of the implantable medical device system. The outer side (host vasculature inter-facing) of the PTFE gradient membrane enables cellular ingress (greater than 1 micron to about 15 micron), and the PTFE gradient membrane titrates down in relative pore size to an appropriate size that would prohibit cellular ingress (about 0.1 micron to about 1 micron) into the cell-containing inner chamber of the implantable medical device. The pore size of the PTFE gradient membrane renders the implantable medical device immuno-isolating for the implanted cells.

In further embodiments, implant systems comprise a surface with an electrospun PTFE gradient membrane combining immunoisolation and vascularization features as described above are provided. An electrospun PTFE multielement layer comprises relatively larger fibers, of a size sufficient to inhibit fibroblast layer formation. This feature may take the form of a final, outer gradient layer comprising multiple strands to form thick fibers of about 25 to about 200 micron in diameter. With such larger fibers randomly oriented on the outer surface of the gradient membrane, the layer serves as a surface to inhibit fibroblasts from forming a fused fibrotic layer.

In another aspect, a manufacturing process and/or method is provided for producing an implantable medical device comprising an immune-isolation chamber of live cells. In one embodiment, the method comprises a series of steps that provide for application of a single layer gradient membrane, such as an electrospun PTFE single layer gradient membrane, to a surface of the implantable medical device. The method can also provide a single step electrospun deposition process wherein a material, such as PTFE, is extruded onto a surface in a manner such as to create increasingly less dense and therefore larger pore size, regions in the single layer membrane plus a modification to the gradient membrane that will form large diameter (about 25 micron to about 200 micron) randomly oriented fibers on the surface of the gradient pore membrane that assist in preventing the formation of tight layers of fibroblasts in the host tissue region close to the implantable medical device/tissue interface. FIG. 2A shows a representation of the gradient membrane with the large pore surface that induces vascularization facing up. Although the 10 micron to 15 micron pore surface will induce the formation of close vascular structures, areas of fibroblast layering can form above the developing vascularized interface. FIG. 2B shows the gradient membrane with a random network of large diameter fibers anchored to the top of the gradient surface. These fibers serve to break up any layer of closely packed fibroblasts that may start to form and will further allow additional vascular structures to form. The fibers may be a non-woven mesh such as polyester or they may be made of electrospun PTFE fibers cast parallel to each other to form relatively larger diameter fibers. Such fibers can be made as a separate network of random fibers and then applied to the gradient membrane or, in the case of electrospun gradient membranes, the electrospinning process can be programmed to switch to a different mode of laying down fibers once the thickness of the gradient membrane has been reached. The new mode of electrospinning creates relatively larger fibers at the surface of the gradient membrane that are contiguous with, or non-contiguous with, the gradient membrane.

In some embodiments, methods of the present disclosure do not require, and advantageously eliminates an assembly step for sealing two separate component membrane layers together. Prior constructs required a separate step of this nature to achieve the fabrication of a membrane coating having varying pore size. The present single layer gradient membranes are absent a sharp demarcation zone within the membrane separating areas or regions of differing pore size.

Various embodiments of the present disclosure contemplate the provision of membranes of the present disclosure on an implantable device. The outer membrane region of the membrane may be further defined as having a surface that is closest to the exterior of the membrane, and would be expected, in some embodiments, to interface with the in vivo environment of an animal or human when provided on the surface of an implantable medical device. The inner membrane region of the membrane is further defined as having a surface that is closest to the interior of the membrane, and in some embodiments forms an interface with a surface or an internal lumen of an implantable medical device. Such an internal lumen would be designed to contain living cells or a therapeutic agent. A transitional gradated membrane region resides between the inner membrane region and outer membrane region in some embodiments of the present disclosure.

In some embodiments, the inner membrane region comprises a gradient of relatively smaller pore size, such as a gradient of from about 0.1 to about 1 micron pore size. In some embodiments, the outer membrane region is characterized as a having a gradient of relatively larger pore size, such as a gradient of from about 2 micron to about 100 micron (or about 5 to about 15 micron). In this embodiment, the transitional gradient membrane region between the inner and outer region is characterized as having a gradual gradient of pore size of between about 1 micron at an interface closest to the inner membrane region, and about 5 micron at an interface closest to the outer membrane region.

In some embodiments, a single layer electrospun gradient membrane is provided that further includes a gradient membrane region having a pore size of between about 15 and about 50 micron at a region closest to an interface with the outer membrane region as described above, or alternatively a gradient pore size of up to about 190 micron.

In some embodiments, the membrane is further defined as a single layer immuno-isolation electrospun PTFE gradient membrane, the single layer membrane comprising gradient individual membrane regions within the single layer, one membrane region having a graduated pore size of about 0.1 to about 1 micron, a membrane region having a pore size of about 2 micron to about 100 micron (or about 15 micron), and a transitioning membrane region there between having a gradient pore size of about 5 micron to about 50 micron (or alternatively between about 5 micron to about 15 micron).

The single layer membrane can be constructed to further include an outer layer comprising a woven or non-woven layer. This outer layer may or may not be attached to the single layer gradient membrane. This layer may comprise a non-woven polyester fiber mesh, or be fabricated to include thicker fibers comprising a non-woven mesh. The outer layer would comprise a pore size greater than about 200 micron. In some embodiments, the outer layer comprises randomly dispersed strands of electrospun polymeric material, such as PTFE, or a non-woven immune-compatible material as polyester.

In another embodiment, an immuno-isolation implantable medical device is provided that comprises a surface having thereon the single layer immuno-isolation electrospun gradient membrane as described herein. This single layer immuno-isolation electrospun gradient membrane may comprise electrospun PTFE, and the single layer immune-isolation electrospun gradient membrane will comprise an inner and an outer membrane region having a gradient pore size. The membrane regions, for example, may comprise a first innermost PTFE membrane region having a gradient pore size ranging from between about 0.1 to about 1 micron, an outer gradient PTFE membrane region having a pore size ranging from about 5 micron to about 50 micron (or about 5 to about 15 micron), and a transition region having a gradual gradient pore size of about 1 micron to about 15 (or 10) micron.

In some embodiments, an immuno-isolation implantable medical device is provided that comprises an inner lumen, and the inner lumen comprises a population of live cells or therapeutic agents. By way of example, the live cells may comprise human cells, such as islet cells, naturally occurring primary cells, cell lines, genetically engineered cells, stem cell derived cells, or a combination thereof.

In some embodiments, the single layer gradient membrane is provided over the entire surface of an implantable medical device.

In yet another embodiment, a method of manufacture of a single layer immuno-isolation electrospun gradient membrane comprising a polymeric material is provided. This single layer immuno-isolation electrospun gradient membrane comprises membrane regions having a gradient pore size produced in a single layer by an electrospinning process, wherein a single membrane layer is created having several gradient membrane regions of different pore size so as to create a continuous and gradual gradient of increasing pore size through the single layer membrane. In one embodiment, the single layer will have an inner membrane region having a gradient pore size of about 0.1 to about 1 micron, an outer membrane region having a gradient pore size of about 5 micron to about 50 (or alternatively about 5 micron to about 15 micron); and a transition membrane region there between having a gradient pore size of about 5 micron to about 40 micron (or alternatively about 5 micron to about 10 micron).

The single layer immuno-isolation electrospun gradient membrane preferably comprises a relatively thin thickness. In some embodiments, the thickness of the single layer gradient membrane is between about 20 micron and 150 micron or any subrange between 20 and 150 micron. The single layer immuno-isolation electrospun gradient membrane does not comprise an abrupt demarcation between the various gradient inner and outer membrane regions or at the interface with the transition membrane region. The continuous gradient of pore size though out the single layer gradient membrane structure presents superior and more uniform diffusion properties, and facilitates a more predictable and steady release of therapeutic agents and compounds that may be included within a lumen of an implantable medical device comprising the single layer gradient membrane. Such features present significant advantages and avoids the problems associated with prior implantable structures, such those structures described in U.S. Pat. No. 6,060,640.

In various embodiments, the present disclosure provides implantable devices having a number of improved characteristics and features. In some embodiments, an implantable device is provided that possesses a unique configuration that facilitates a maximization of surface area available for vascularization by a host animal. The configuration of the implant device, in some embodiments comprises a multi-component structure, comprising one or more individual element members and a hub and/or a manifold, wherein the individual pod elements are in communication with the hub and/or manifold. In this regard, means are provided that permit multiple of the individual element members of the device to communicate with at least one common component of the device, such as a hub or a manifold. In this manner, and where the individual member element comprises an internal lumen, access to the lumen of each individual element member and the hub and/or manifold is provided.

Implant devices of the present disclosure comprise unique configurations and may be implanted in a manner that optimizes the number of devices per unit area of a surgical site in a patient. The configuration of the implantable device can be optimized to the shape and size of a particular surgical site into which it is being inserted into a patient, such as to closely pattern the surgical insertion site created by a blunt tissue dissection. The design of the individual element members of the implantable device also permits enhanced access to the interior lumen areas of the element members, making the device readily available to addition of an agent of interest suitable for delivery to a host, such as a therapeutic agent, or alternatively, to the loading of a live cell population to the lumen.

In various embodiments, implantable devices of the present disclosure comprise a manifold having a means to provide communication from one or more element members (i.e. immuno-isolation devices) to a hub of the device. The means to communicate between the manifold and an element member may be implemented to selectively transfer oxygen, therapeutic agents, nutritional agents, electrical signals, electrical power or multiple combinations thereof to the element member. In certain embodiments, communication means from the manifold to the element member(s) comprise a tube or catheter to supply gas or liquids to the element member, such as specifically to a lumen of an element member. This connecting communication means may also be utilized as part of the implant device to connect electrical wires or circuit leads to transmit electrical signals or power, or to communicate combinations of materials to the lumen of the element member.

In some embodiments, the hub or central portion of an implant comprises a component within which the implant device may house an oxygen generator, pump for therapeutic agents or nutritional agents, reservoir(s), electronics, power supply or combinations thereof, and to communicate to element members via the manifold.

The manifold and hub of implant devices of certain embodiments impart a number of distinct functions to the device. For example, the manifold provides a pathway to communicate between the element member (immuno-isolation device) and a hub. The hub, in some embodiments, provides a structure in which functional elements of the implant device may be housed. In some embodiments, the implant device comprises both a manifold and a hub, and the manifold is in communication with the hub. Configurations of the device implant are also provided where an element member is in communication with more than one hub and a (or more than one) manifold, such as through one or more connection means between the manifold and the lumen of an element member. In some embodiments, the implant device will comprise element members having multiple access ports and lumens.

In some embodiments, the hub and/or manifold comprises a surface which comprises a vascularizing material. By way of example, such a vascularizing material may comprise an immune-isolating membrane, for example, a 5μm nominal pore size expanded PTFE membrane. This membrane serves to reduce the inflammatory response of a host once the implant device is provided under the skin (subcutaneously) in the animal.

The advantages of the presently disclosed immune-isolation implantable devices include a maximization of surface area presented by the device available for vascularization by a host. In particular, implantable devices or portions thereof that comprise an immuno-isolation device present surface area that may be vascularized by the host when implanted. This structure maximizes vascularization of the device as a whole in the animal. Implantable devices of the present disclosure comprise at least one manifold and a hub, the manifold being in communication with one or more pod members. Pod members comprise at least one lumen providing a communication pathway. In some embodiments, each lumen comprises at least one distinct chamber within the lumen.

In some embodiments, pod elements of the present disclosure are (i) tapered at the proximal end to minimize the overlap of multiple implant devices in communication with the manifold, (ii) tapered at one end to enable multiple pod elements having a lumen to be implanted with at least one cross-section surface of the pod element (and the lumen contained therein) to be in contact with the in vivo host environment upon implantation, at a single surgical site, (iii) tapered at one end to minimize the distance from any adjacent pod member, (iv) shaped to have an overall shape that is similar to that created by a common blunt surgical instrument during an implantation procedure, (v) shaped to optimize and minimize the length of the communication means (such as a tube or catheter) that is provided to establish access and/or communication between the manifold and a pod member, or two or more pod members implanted in a single surgical site.

The multi-component implantable device may be further described as an immuno-isolation device. In some embodiments, each pod member comprises a tapered end having at least one access port in communication with at least one lumen of a pod. The taper enables multiple devices to be implanted (i) in a stack one-on-top-the-other configuration, (ii) edge to edge in a fan configuration, (iii) overlapping to expose a portion of the top and bottom to the in vivo environment of the host. At least one proximal port of each pod member may be in communication with a manifold, so as to provide access of the manifold to the lumen of each pod member. Other ports can be located at each element member of the immune-isolation device. These additional ports may be used to facilitate additional access to the lumen of the pod member. The individual pod members and their internal volumes are filled with an identified amount of desired cells or therapeutic agents. The desired cell population, for example, may comprise cells that are designed to secrete a therapeutic product. By way of example, the cells may comprise a population of cells enriched for islet cells capable of secreting insulin through the membrane of the lumen and into the in vivo environment of the host, in response to circulating glucose levels in the host. Alternatively, the chambers may be empty and a drug may be introduced through injection or pumping into a hub for distribution to the multiple attached chambers.

In various embodiments, one or more pod members of immuno-isolation devices of the present disclosure comprise an electro-chemical or optical sensor provided in communication with the hub and the manifold. Communication means of the manifold including, but not limited to, electrical wiring, pumps, and other features, are operable to transmit power, a pre-pulse signal, a measurement signal, and/or oxygen to and from the sensor. A pre-pulse signal is contemplated at least in embodiments comprising electro-chemical sensors to initiate a measurement. Devices of the present disclosure comprising one or more pod members and porous membranes provide means to transport fluids or agents from vascular structures adjacent to a device surface to the encapsulated sensor.

Alternatively, one or more lumens of the present disclosure are operable to disperse one or more therapeutic agents to a host. For example, a lumen of pod may be provided with an active agent, such as an active biological agent, insulin, Factor VIII, Factor IX, HGH hormone, or proteins from the hub via the manifold. The active agent will then be released through the lumen of the pod of the implant device and be rapidly dispersed through the vascular structures formed surrounding the implant device. In the above manner, and through an interconnection of the pod ports, the immune-isolation device implanted into the soft tissue of an animal, such as a human, may also be configured to communicate with other implanted immune-isolation devices, device manifolds, catheters, or other desired materials through one or more of the available device pod ports.

In one embodiment, an immuno-isolation membrane is provided that comprises an inner region and an outer region. The inner region and the outer region each comprise pores with a pore-size gradient from the inner region to the outer region. The inner region comprises a pore size of between about 0.1 micron and about 1.0 micron, and the outer region comprises a pore size of between about 3.0 micron and about 15 micron. In various embodiments, methods of forming and manufacturing membranes and devices are provided. In one embodiment, a method of manufacturing an immuno-isolation membrane comprising an inner membrane region, an outer membrane region, and a transition gradient region there between. The method comprises steps of depositing an electrospun inner membrane region, wherein the inner membrane region comprises a porous structure with pore sizes of between 0.1 micron to 1.0 micron; depositing an electrospun outer membrane region, wherein the outer membrane region comprises a porous structure with pore sizes of between 2.0 micron to 50.0 micron; and wherein the inner membrane region and the outer gradient membrane region are formed with a continuous pore size gradient devoid of lamination or welding between the regions.

In one embodiment, an implantable medical device operable for subcutaneous implantation in an animal is provided wherein the device comprises a hub comprising an internal void, and at least one pod in communication with the hub. The pod comprises an inner cavity operable to receive at least one of cells, a gas and a therapeutic agent. An immuno-isolation member is provided adjacent to and exterior to the inner cavity. A vascularizing membrane is provided adjacent to and exterior to the immuno-isolation member. The hub and the pod are provided in communication with one another by at least one channel extending between the internal void of the hub and the inner cavity of the at least one pod.

In one embodiment, an implantable medical device is provided that is operable for subcutaneous implantation in an animal. The device comprises a hub with an internal void, and at least one pod in communication with the hub. The pod comprises an inner cavity operable to receive at least one of cells, a gas and a therapeutic agent. An immuno-isolation member is provided adjacent to and exterior to the inner cavity, and a vascularizing membrane provided adjacent to and exterior to the immuno-isolation member. The vascularizing membrane comprises an inner region and an outer region, the inner region and the outer region each comprise pores, and a pore-size gradient is provided from the inner region to the outer region. The inner region comprises pore sizes of between about 0.1 micron and about 2.0 micron, and the outer region comprises pore sizes of between about 2.0 micron and about 20 micron. The hub and the pod are provided in communication with one another by at least one channel extending between the internal void of the hub and the inner cavity of the pod.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In addition, the materials, methods, and examples are illustrative only and are not intended to be necessarily limiting.

### DESCRIPTION OF THE DRAWINGS

FIG. 1A is an elevation of an electrospun membrane structure according to one embodiment of the present disclosure.

FIG. 1B is an elevation view of a laminate membrane structure according to the prior art.

FIG. 2A is a perspective view of an electrospun membrane according to one embodiment of the present disclosure.

FIG. 2B is a perspective view of an electrospun membrane according to one embodiment of the present disclosure.

FIG. 3 is a plan view of a component of an implantable immune-isolation device according to one embodiment of the present disclosure.

FIG. 4 is a plan view of a component of an implantable immune-isolation device according to one embodiment of the present disclosure.

FIG. 5 is a cross sectional view of component of an implantable device according to one embodiment of the present disclosure.

FIG. 6 is a cross-sectional elevation view of a component of an implantable device according to one embodiment of the present disclosure.

FIG. 7 is a plan view of an implantable device according to one embodiment of the present disclosure.

FIG. 8 is a side elevation view of the implantable device according to the embodiment of FIG. 7.

FIG. 9 is a perspective view of the implantable device according to the embodiment of FIG. 7.

FIG. 10 is a cross-sectional elevation view of a component of the implantable device according to FIG. 7. FIG. 11 is an elevation view of an implantable device according to one embodiment of the present disclosure implanted in a patient.

FIG. 12 is a detailed cross-sectional view of a portion of an implant according to one embodiment of the present disclosure.

FIG. 13 is a detailed cross-sectional view of a portion of an implant according to one embodiment of the present disclosure.

FIG. 14 is a detailed cross-sectional view of a portion of an implant according to one embodiment of the present disclosure.

### DETAILED DESCRIPTION

Reference to an element by the indefinite article “a” or “an” does not exclude the possibility that more than one element is present, unless the context clearly requires that there be one and only one element. The indefinite article “a” or “an” thus usually means “at least one.”

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

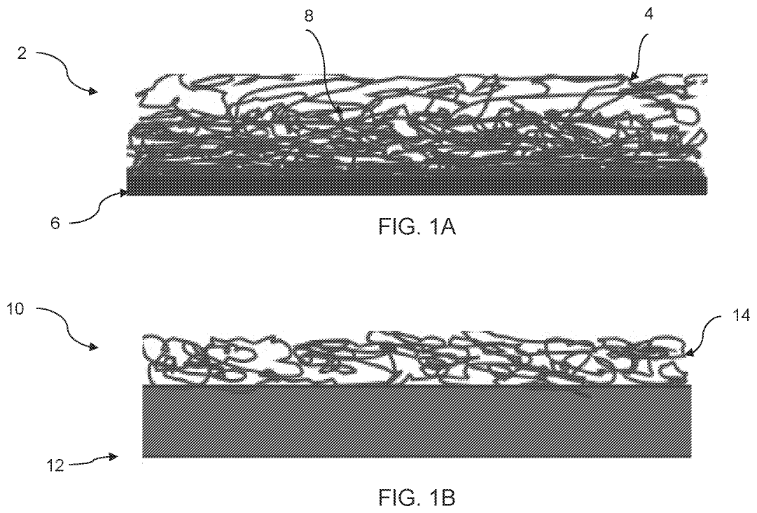

FIG. 1A is an elevation view of an electrospun PTFE membrane 2. The membrane 2 is contemplated for use in certain embodiments to provide a chamber with a lumen to hold living cells. The outermost layer or region 4 of the membrane 2 comprises a plurality of randomly arranged polymeric strands creating relatively large pore spaces on the order of between approximately 5 to 50 micron. In some embodiments, pore sizes at or proximal to the outer region 4 of the membrane are approximately 15 micron. An innermost surface 6 of the membrane structure 2 comprises a plurality of tightly woven PTFE fibers creating pore sizes of about 0.1 to 1.0 micron. A gradient of pore sizes exists between the innermost region 6 and the outermost region 4, wherein density gradually decreases when travelling from the innermost to outermost region. The membrane 2 of FIG. 1A comprises a single-layered element with a pore-size gradient and wherein a portion 6 of the membrane 2 is operable to act as an immuno-protective area and an outer portion 4 of the membrane 2 is operable to act as a vascularizing structure.

FIG. 1B illustrates a non-gradient membrane 10 comprising two distinct layers 12, 14. A first layer 12 comprises a membrane having small pores throughout of about 0.1 to 1 micron that is operable to serve as an immuno-protective layer. A second layer 14 comprises an outer membrane of material having pores of about 5.0 to about 10.0 micron and is operable to serve as a vascularizing layer. FIG. 1B illustrates a known membrane structure essentially comprised of two layers of two different pore sizes. The layers 12, 14 are laminated, adhered, or otherwise connected to one another.

Typically, implantable immuno-isolation medical devices suitable for carrying a chamber of live cells are constructed with a vascularizing membrane and are assembled using two separate layers made separately, and then joined together (FIG. 1B). Embodiments of the present disclosure, including the membrane 2 of FIG. 1A, provide for a gradient membrane 2 manufactured by electrospinning a polymeric material into a mat of randomly stacked fibers 8. In an initial phase of manufacture, fibers 8 are collected after emerging from a spinneret (syringe or nozzle, not shown), and the fibers 8 are directed to a collector under high voltage where they stack and cross each other with very close spacing such that a tight mat-like structure is formed. This produces a layer of small pores that will not allow cells of the immune system to penetrate through this first region 6 of the membrane structure. The equipment ejecting the continuous fiber is programmed and operable to gradually deposit a less dense series of overlapping and randomly organized strands of fibers 8 so that the effective pore size between strands starts to open creating larger pores. The process continues until the desired thickness of the mat of fibers is reached and the nominal pore size is about 10 micron to about 15 micron. FIG. 1A shows a tight mat-structure at the bottom region 6 of the membrane 2 and a more open structure created in gradient fashion with the final large pore of about 10 micron to about 15 micron reached at the top surface 4.

By contrast, FIG. 1B depicts known structures formed by laminating two separate membranes 12, 14 to create a composite 10. The bottom membrane 12 comprises a tight pore, dense structure that prevents cell passage through the membrane. The top layer 14 comprises an open structure that will allow cell penetration up to the lower dense layer. The two membranes are laminated together by an adhesive or by sintering of the membranes. Nevertheless, the structure is prone to peeling or delamination thus adversely affecting the function of the laminate membrane. Where the two layers 12, 14 are joined, an abrupt transition zone is formed. These two layers provide an outer layer 14 (vascularizing layer) and a separate inner, more dense immune-isolating membrane layer 12.

Advantageously, and according to methods of the present disclosure, a single layer membrane 2 is constructed that allows for cell penetration to a certain extent and which is not prone to delamination. Embodiments of the present disclosure provide a single layer with a first gradient region that allows for vascularization, and a second gradient region (an inner region) providing for a more tightly woven electrospun membrane (such as a PTFE membrane). Such single layer membrane of varying porosity with a gradient of pore sizes are contemplated as being formed in-place on an implantable medical device including, but not limited to, those shown and described herein.

In certain embodiments, a single layer gradient membrane is constructed separately and then provided to a desired surface of an implantable medical device during manufacture of the implantable medical device, such as by application of a sheet of a pre-fabricated single layer gradient membrane as described herein to the desired surface or surfaces. Notably, the present single layer gradient membranes do not have an abrupt transition zone within the membrane, as is characteristic of other bi-membrane systems. Electrospun membranes of the present disclosure serve as a single component embodying immunoisolation and vascularization features and comprise a thickness of at least about 20 micron and not more than about 200 micron. In some embodiments, at least two membranes are contemplated as being provided and welded together in a manner that creates or defines an interior cavity that is operable to receive therapeutic agents including, but not limited to cells. The surface of the membrane 2 facing or provided adjacent to the interior cavity comprises tightly intertwined fibers that create pores from about 0.1 micron to about 1 micron. A continuous transition in gradient is provided from this tight intertwined structure to a more open or loose intertwined fiber network and as one progressed from the inner structure to the outer surface, and the transition is to a more and more open structure in a gradual gradient. Likewise, the pores gradually transition from about 0.1 to about 1 micron at the inner surface 6 facing the lumen to between about 5 and 50 micron, and preferably of about 10 to about 15 micron towards the outer surface of the membrane 4.

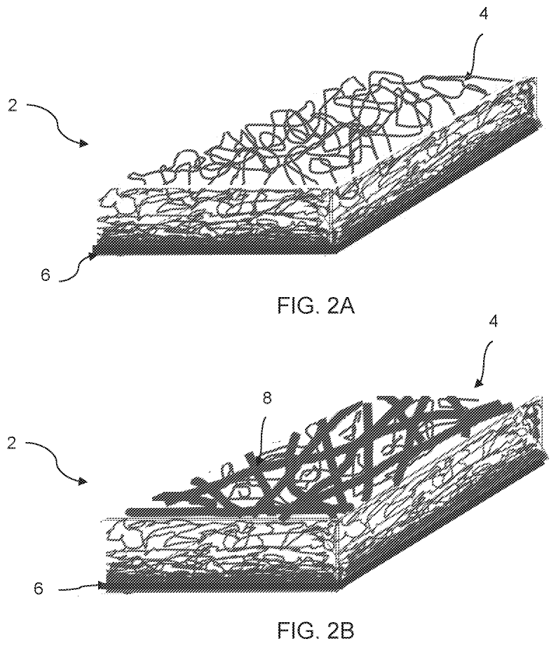

FIG. 2A is a perspective view of a section of a single component gradient membrane 2 where the outer surface 4 comprises an electrospun material of pore sizes of about 5 to about 50 micron, and preferably of about 5 micron to about 15 micron.

FIG. 2B is a perspective view of a section of a single component gradient membrane 2 having large strands of electrospun fibers 8 randomly oriented along the surface. Alternatively, the fibers 8 may comprise a non-woven mesh of a polymeric material randomly oriented along the surface. Such randomly oriented strands may be about 25 micron to about 200 micron in diameter.

FIG. 2A illustrates a pore gradient membrane 2 that prevents or minimizes the formation of tight layers of fibroblasts in the host tissue region close to an implantable medical device/tissue interface. FIG. 2A shows the pore gradient membrane 2 with the large pore surface that induces vascularization at an upper region 4 of the membrane. The outer region 4 preferably comprises a pore structure with pores of between approximately 10 to 15 micron, and the pore structure induces the formation of close vascular structures. Areas of fibroblast layering can form above the developing vascularized interface. FIG. 2B shows the gradient membrane 2 with a random network of relatively larger diameter fibers 8 anchored to the top of the gradient membrane surface 4. These larger fibers 8 break up any layer of closely packed fibroblasts that may start to form in the area of implantation and will further allow additional vascular structures to form. The larger fibers 8 are contemplated as comprising a non-woven mesh, such as polyester, or as comprising electrospun PTFE fibers cast parallel to each other to form larger diameter fibers. Such larger fibers can be made as a separate network of random fibers and then applied to the gradient membrane. Alternatively, in the case of an electrospun gradient membrane, a layer of relatively larger fibers may be provided to overlay the gradient membrane by using an electrospinning process. This may be achieved by programming an electrospinning device to switch to a different mode or pattern of laying down fibers, once the desired thickness of the gradient membrane has been reached. This new mode or setting of electrospinning can therefore be used to create large fibers at the surface of the gradient membrane, this larger fiber containing component therefore being provided as contiguous with the underlying gradient membrane.

In certain embodiments, an implantable medical device having multiple components is provided wherein one of the components, for example, a lumen chamber suitable for containing a population of live cells (e.g., stem cells or other desired material) having an immune-isolating membrane, may be processed to include the single layer gradient membrane described herein over all or a portion of the implantable medical device. Such would provide surfaces suitable for enhancing vascularization to the implantable medical device in vivo. A sonic welding technique may be used, for example, to apply and secure the single layer gradient membrane to the surfaces of the implantable medical device. In various embodiments, electrospun membranes are provided that comprise strand 8 sizes of about 5 micron or less, 5 micron pore sizes and a preferred thickness of between about 5 and 1,000 micron, and more preferably of about 15-90 micron. A gradient is provided wherein a pore size of a membrane is between approximately 5 to 15 micron proximal to an outer portion of the membrane, and decreases to about 0.4 micron or less at an inner portion of the membrane.

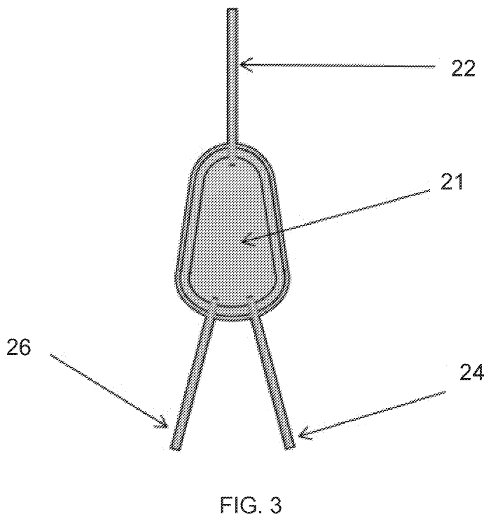

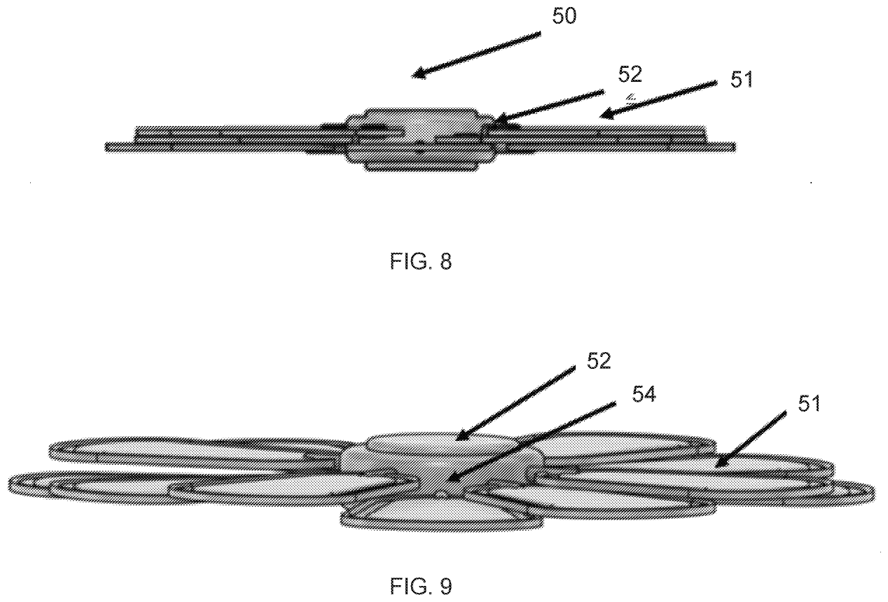

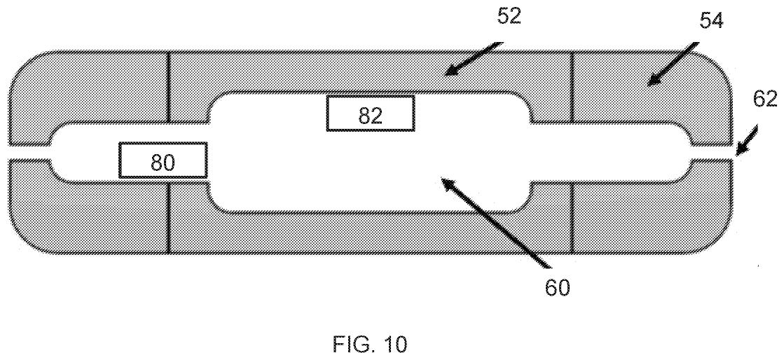

FIGS. 3-11 depict implantable medical devices and portions thereof. Embodiments of the present disclosure include, but are not limited to, implantable medical devices that comprise membranes 2 of the present disclosure as a component thereof. The depicted devices are suitable with gradient membranes of the present disclosure including, for example, those shown in FIG. 1B. It will be recognized, however, that devices of the present disclosure including those shown in FIGS. 3-11 are not limited to, and need not necessarily be combined with membrane structures.

FIG. 3 is a plan view of a feature of an implantable immune-isolation device 20. As shown, the device 2 comprises a plurality of ports 22, 24, 26. A first port 22 is provided in communication with at least one lumen or pod 21 of the immuno-isolation device 20. Access to the device lumen either pre or post implant is achieved by connecting to at least one of a hub (not shown in FIG. 3) and a port. In this embodiment, the distal ports facilitate access to one or more pods.

Preferred methods for creating a side seal or peripheral of the device include but are not limited to ultrasonic welding, heat sealing, over-molding, gasket compression, compression, silicone, glue, spin welding, laser welding, and various combinations thereof. In some embodiments, polyethylene inserts are provided, which are melted and driven into a perimeter or periphery of the device to create a seal around the pod. In some embodiments, the side or peripheral seal also secures the ports 22, 24, 26 to the pod 21. U.S. Pat. No. 5,545,223 to Neuenfeldt et al. discloses devices and methods for sealing implants, and is hereby incorporated by reference in its entirety.

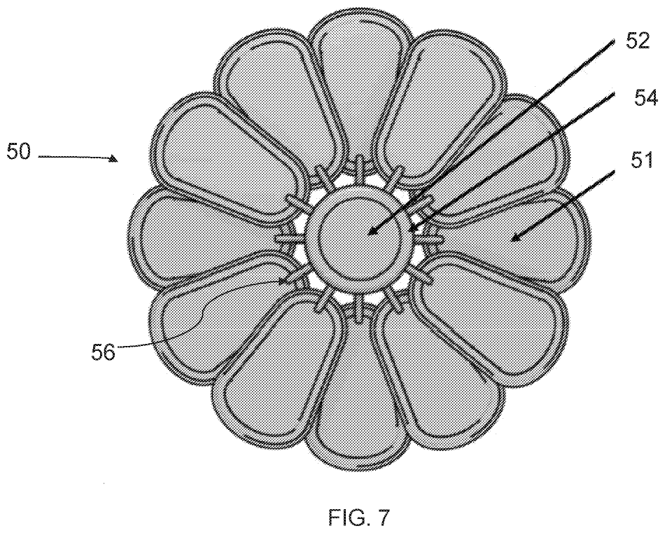

As shown in FIG. 3, a pod element 21 of the present disclosure comprises a tapered shape to facilitate insertion of the device into tissue while also maximizing surface area and internal volume of the pod 21. An internal volume of the pod 21 is provided in communication with internal channels of each of the ports 22, 24, 26. In some embodiments, one or more of the ports 22, 24, 26 comprises 28 gauge tubing and wherein a first end of the port(s) is in communication with the pod 21 and the second end of the port is in communication with a central member or hub (see FIG. 7, for example).

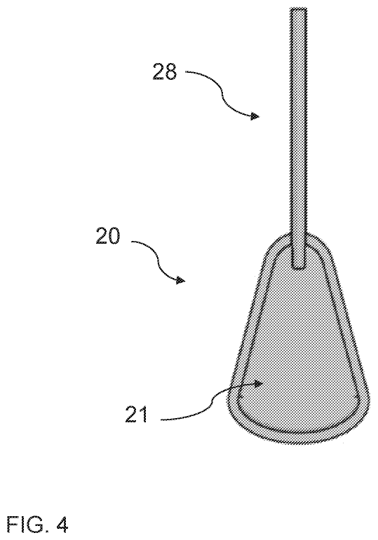

FIG. 4 is a plan view of a portion of an implantable device 20 according to another embodiment of the present disclosure wherein the device 2 comprises a single port 28. As shown, the port is in communication with at least one pod 21 of the immuno-isolation device.

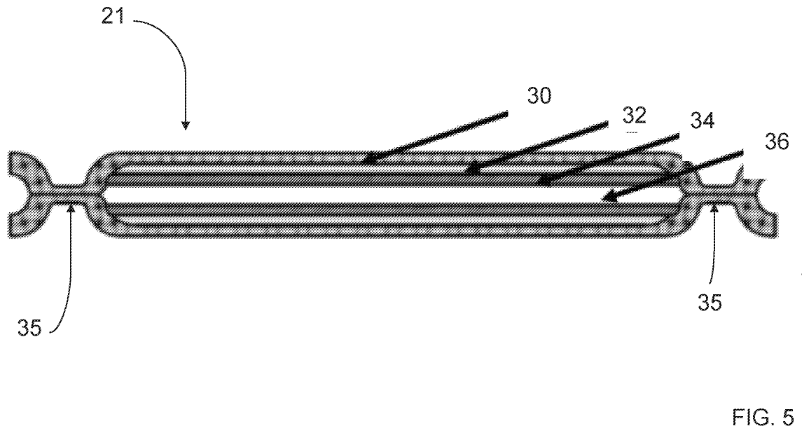

FIG. 5 is a cross-sectional elevation view of a pod 21 comprising a single lumen or interior cavity. The housing or pod 21 comprises an outer layer 30, which preferably comprises a porous material and a vascularizing structure 32 within the outer layer 30 to induce vascularization at the surface and to reduce the immune response by a recipient. An immune barrier 34 is provided within the vascularizing structure 32 to prevent ingress cells from the recipient to the lumen or inner cavity 36 of the pod 21. In certain embodiments, the vascularizing structure 32 and the immune barrier or immuno-protective layer 34 comprise distinct layers (see FIG. 1B, for example). In alternative embodiments, the vascularizing structure 32 and the immuno-protective barrier 34 comprise a single element with different regions having different properties. For example, the vascularizing structure 32 and the immuno-protective barrier 34 are contemplated as being provided by the membrane 2 of FIG. 1A. Accordingly, the vascularizing structure 32 and the immuno-protective barrier 34 do not necessarily comprise two discrete layers or elements, and are contemplated as comprising a single layer with a pore-size gradient and wherein the layer is operable to provide both an immuno-protective barrier and a vascularizing structure.

FIG. 5 also shows a peripheral seal 35 extending around the device. The seal 35 may be formed by sonic welding, for example, and generally provides a seal and structure to the device 21.

In various embodiments, a pod 21 with an internal volume or void comprises a peripheral seal formed by one or more of ultrasonic welding, heat sealing, over-molding, gasket compression, compression, silicone, glue, spin welding, and laser welding. In various embodiments, the outer porous structure 30 comprises a porous surface area over at least about 20% of the surface area of the structure 30. The vascularizing structure 32 comprises a porous structure with pores of between approximately 0.1 μm to 50 μm in diameter. The immune barrier 34 comprises a porous structure with pores of less than approximately 1.0 μm in diameter. The inner cavity 36 comprises a void to house or receive cells, tissues, therapeutic agents, oxygen, sensors, nutrients, pumps, electronics, electrical connectors, or combinations thereof.

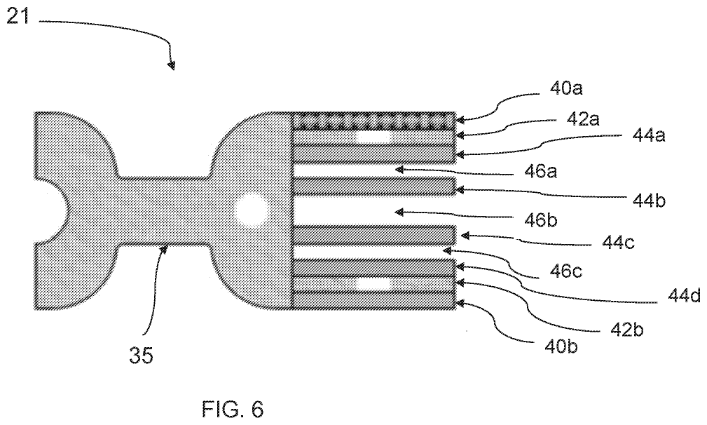

FIG. 6 is a partial cross-sectional elevation view of a pod 21 according to one embodiment of the present disclosure. As shown, the pod 21 comprises a plurality of layered elements. Specifically, the pod 21 comprises a first outer polyester woven mesh layer 40a.

A first vascularizing membrane 42a is provided within the outer layer 40, and a first immuno-isolation member 44a is provided within and adjacent to the first vascularizing membrane 42a. A first lumen or interior void 46a is provided within the first immuno-isolation member 44a and a second immuno-isolation member 44b. A second interior void 46b is provided between the second immuno-isolation member 44b and a third immune-isolation member 44c. A third interior void 46c is provided between the third immune-isolation member 44c. A fourth immune-isolation layer 44d is provided, and is adjacent to a second vascularizing membrane 42b. A lower portion of the device (at least as shown in FIG. 6) comprises a second polyester mesh layer 40b. The layers and the device 21 are secured by and provided with a side seal 35 which, in various embodiments, is formed by at least one of ultrasonic welding, heat sealing, over-molding, gasket compression, compression, silicone, glue, spin welding, laser welding, and various combinations thereof.

As shown and described with respect to FIG. 5, the embodiment of FIG. 6 (and other embodiments of the present disclosure) are contemplated as comprising an immuno-protective layer (44a, for example) adjacent or proximal to a vascularization layer (42a, for example). The immuno-protective layer(s) and the vascularization layer(s) may comprise a single element with different properties (such as the membrane of FIG. 1A, for example) or, alternatively may comprise separate layers that are formed, connected, or adhered together or simply provided adjacent to one another (such as the membrane of FIG. 1B, for example).

FIG. 6 depicts a device comprising three interior cavities or voids operable to house and receive materials. In some embodiments, it is contemplated that devices in accordance with the embodiment of FIG. 6 further comprise and are provided with oxygen gas in the central void 46b, and the additional interior void members 46a, 46c comprise cells. A plurality of membranes 44a, 44b, 44c, 44d preferably comprise a PTFE with pores of about 0.4 micron. The vascularizing membrane layers 42a, 42b preferably comprise a PTFE electrospun nonwoven polyester mesh layer. Outer woven polyester structures 40a, 40b are provided to give strength and support to the overall pod structure 21.

Although interior void members 46 are described as comprising a void or lumen, it will be recognized that these regions are contemplated as receiving materials and may not necessarily comprise a “void” upon complete assembly of the device. The interior void members 46a, 46b, 46c are contemplated as comprising cells, gas, and/or various therapeutic agents. Additionally, in some embodiments, one or more of the interior void members 46a, 46b, 46c are contemplated as comprising or receiving one or more of a pump, a sensor (e.g. oxygen sensor), power storage (e.g. a battery), and electronics (e.g. a controller). The foregoing is true for lumens and interior voids of various embodiments of the present disclosure and is not limited to the embodiment of FIG. 6 which depicts three separate interior void spaces.

FIGS. 7-9 depict an implantable medical device 50 according to one embodiment of the present disclosure. As shown, the device 50 comprising a fan-like configuration with a plurality of pods 51 distributed about a hub 52 in a concentric and overlapping manner. Although the embodiment of FIGS. 7-9 depict twelve pods 51 distributed about a hub, it will be recognized that the present disclosure is not limited to any particular number, spacing, or arrangement of pods 51. In preferred embodiments, the pods 51 are in communication with a manifold 54 that extends at least partially around the hub 52. The pods 51 are connected to the hub 52 and manifold 54 via one or more ports 56. The hub, manifold, and pods are provided in fluid communication with one another by passageways or conduits that are operable to transmit fluid. The passageways or conduits are also operable to house or receive mechanical components such as wiring, valves and other features. Implantable devices of the present disclosure, including that shown in FIG. 7, are operable for use as ported immune-isolation devices in patients whom are insulin dependent, patients with hemophilia, patients with cancer, patients with chronic pain, patients with renal disease, patients requiring drug infusion and shunts, patients with cardiovascular disease, patients with electronic implants, and many other long term disease and/or pain management applications of the implants.

The fan-like configuration of the implantable device 50 of FIG. 7 comprises multiple pods 51 having the structure of the pod 21 of FIGS. 4-5, for example, and are contemplated as being implanted subcutaneously in a patient. The interior volumes 36 of the pods 21, 51, in certain embodiments, are provided with living cells that secrete or that are induced to secrete therapeutic molecules. These molecules will then diffuse through the layers of the device (44a, 42a, 40a of FIGS. 6 and 34, 32, 30 of FIG. 5, for example) and into the host's surrounding tissue. In this manner, the therapeutic molecule(s) will be taken up by the surrounding vasculature and more efficiently distributed throughout the host body. Methods of treating patients and administering drug delivery are thus contemplated wherein the methods comprise providing the implantable devices of the present disclosure with at least one of living cells and a therapeutic agent, and thereafter providing the implantable device 50 within a patient subcutaneously. In further embodiments, the device 50 may be provided or replenished with cells or agents subsequent to implantation.

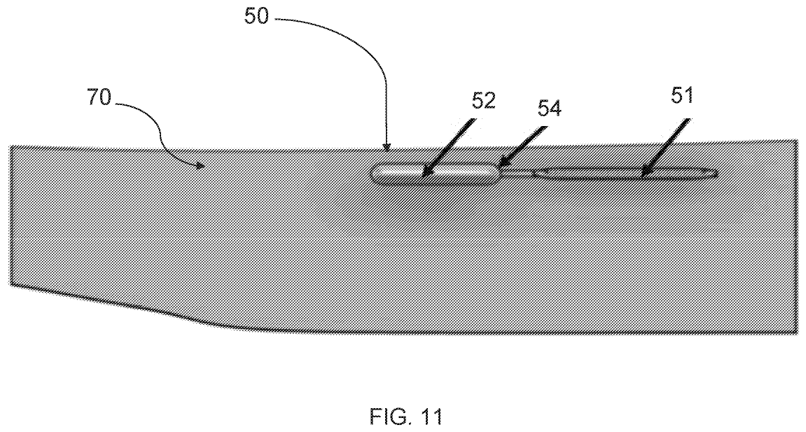

FIG. 10 is a cross-sectional elevation view of a hub 52 and manifold 54. As shown, the hub 52 comprises an internal void 60. The hub 52 provides a housing for various elements including, but not limited to a pump, reservoir, oxygen generator, electronics, power supply, an injection port, and combinations thereof. The manifold 54 comprises a pathway to communicate therapeutic agents, nutrients, oxygen, electrical signals, electrical power, fluids, gases, and combinations thereof from the hub to one or more pods 51 (not shown FIG. 10) via a passage or aperture 62 in the manifold.

In the case of cellular therapies, the pod(s) 21, 51 of an implantable device of the present disclosure are provided with cells that secrete therapeutic molecules intended to treat a disease condition in the patient. Those cells may be primary, natural cells obtained from human donors, an immortalized cell line derived from a specific human tissue, a human cell line derived from tissue that does not produce any therapeutic molecule but has been genetically engineered in the laboratory to secrete a specific protein or stem cell derived tissue in which stem cells have been converted to a specific tissue in the laboratory.

By way of example, in the case of primary tissue that occurs naturally in the body, one might fill the interior volume 36 of a pod with parathyroid tissue harvested from a human donor thereby providing parathyroid hormone to individuals suffering from parathyroid insufficiency.

In various embodiments, it is contemplated that implants of the present disclosure comprise various internal structures and features. For example, and as shown in FIG. 10, at least one of the hub 52 and the manifold 54 of the device 50 comprises a pump 80 and an oxygen sensor 82. Although the pump and the oxygen sensor of FIG. 10 are shown as being within the hub 52 and/or manifold 54, it is also contemplated that components are provided within pods 21 of the present disclosure. Additionally, devices of the present disclosure are not limited to those which comprise pumps and sensors. In addition to or in lieu of pumps and sensors, implantable devices of the present disclosure are contemplated as comprising electronic components and power storage. For example, in some embodiments, electronic components are provided that provide the ability for the implant to communicate with additional, external devices. More specifically, it is contemplated that implantable devices of the present disclosure comprise Bluetooth, RFID, and/or WiFi enabled components that are operable to send and receive signals to a base station or central computer. Such devices may communicate information including, for example, a power level of the device, a fill level of a therapeutic agent (e.g. insulin), and other information.

FIG. 11 is a cross-sectional view of an implantable device 50 comprising a hub 52 with a manifold 54 and a pod 51 extending therefrom. The device 50 is shown as being implanted in the tissue 70 of a patient. In various embodiments, methods are provided wherein the device is implanted subcutaneously and preferably at a depth of less than one inch within an outer dermis layer of the patient such that the device 50 is relatively easy to access for removal, refill, maintenance, etc.

In the case of a cell line, devices of the present disclosure are contemplated as being filled with cells maintained in culture at repositories such as the American Type Culture Collection that express therapeutic proteins. Fibroblast cell lines may be used as a generic cell type for genetic engineering where one or more genes might be inserted by genetic engineering methods to create cells that secrete proteins necessary to treat diseases. Examples include cells engineered to produce Factor IX for a form of hemophilia or erythropoietin for patients with anemia secondary to kidney disease. It is now possible to direct the maturation of stem cells along the pathway to specific cell types. For example, stem cells can be manipulated in the laboratory to convert to pancreatic cells such as B-cells that secrete insulin. Such cells may be loaded into the interior volume 36 of the pods 21 to provide a treatment for diabetes.

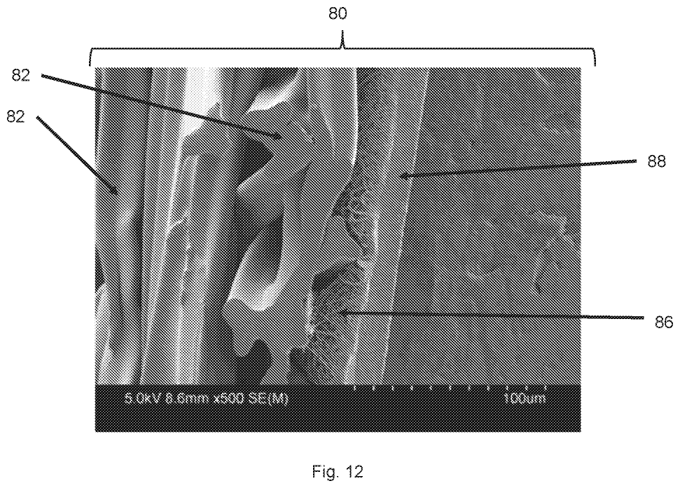

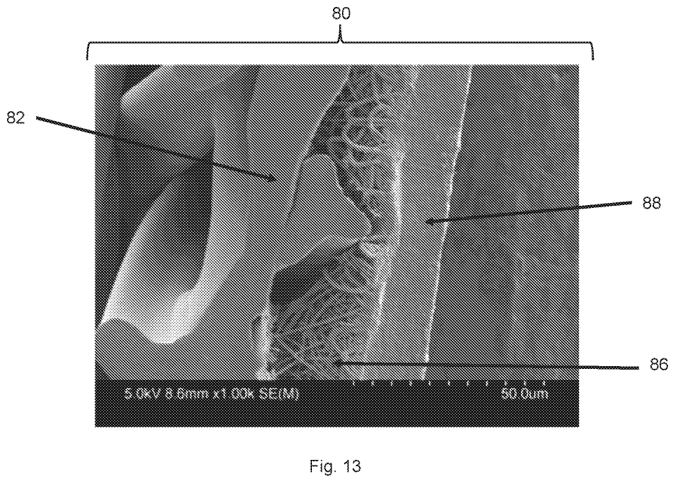

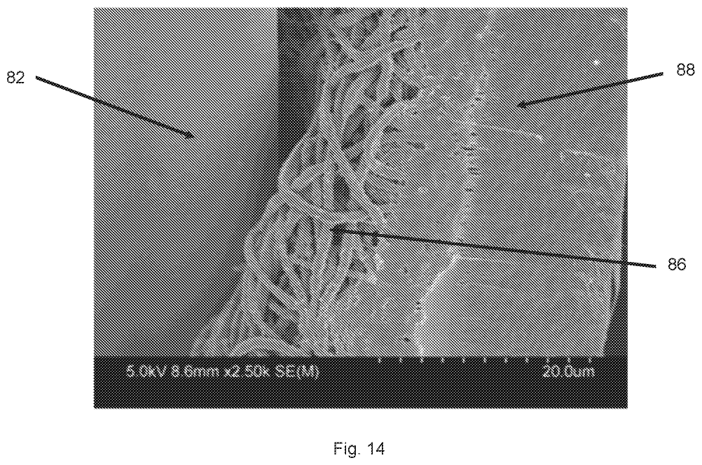

FIGS. 12-14 are detailed cross-sectional views of a portion of an implant 80 according to one embodiment of the present disclosure. FIGS. 12-14 include a scale indicating the approximate distance and size of various depicted components. As shown, the implant comprises a non-woven mesh structural layer 82. A layer of fibers 86 operable to permit vascularization is provided substantially adjacent to the woven mesh 82. In some embodiments, the layer of fibers 86 comprises a pore size gradient that decreases from left to right in FIG. 12. An immuno-protective layer 88 is provided adjacent to the layer of fibers 86. In some embodiments, the layer of fibers 86 and the immuno-protective layer 88 comprise separate layers that are laminated or otherwise adhered together (see FIG. 1B, for example). In other embodiments, the layer of fibers 86 and the immuno-protective layer 88 comprise a single element including, for example, an element formed by electrospinning or otherwise depositing PTFE and wherein the immuno-protective layer comprises an area of smaller pore sizes than the layer of fibers 86 (see FIG. 1A, for example).

The examples set forth above are provided to give those of ordinary skill in the art a complete disclosure and description of how to make and use the embodiments of the methods for prediction of the selected modifications that may be made to a biomolecule of interest, and are not intended to limit the scope of what the inventors regard as the scope of the disclosure. Modifications of the above-described modes for carrying out the disclosure can be used by persons of skill in the art, and are intended to be within the scope of the following claims.

It is to be understood that the disclosure is not limited to particular methods or systems, which can, of course, vary. It is also to be understood that the terminology used herein is for the purpose of describing particular embodiments only, and is not intended to be limiting.

A number of embodiments of the disclosure have been described. Nevertheless, it will be understood that various modifications may be made without departing from the spirit and scope of the present disclosure. Accordingly, other embodiments are within the scope of the following claims.

What is claimed is:

1. An immuno-isolation membrane comprising:
an inner region and an outer region;
wherein the inner region and the outer region each comprise pores with a pore-size gradient from the inner region to the outer region;
wherein the inner region comprises a pore size of between about 0.1 micron and about 1.0 micron, and the outer region comprises a pore size of between about 3.0 microns and about 15 microns.

2. The immune-isolation membrane of claim 1, wherein the membrane comprises a polymeric material.

3. The immune-isolation membrane of claim 1, wherein the membrane comprises a thickness of between about 20 microns and about 150 microns.

4. The immune-isolation membrane of claim 1, wherein the outer region further comprises at least one of a randomly dispersed strands of electrospun polymeric material and a non-woven immune-compatible material.

5. The immuno-isolation membrane of claim 2, wherein the polymeric material comprises polytetrafluoroethylene.

6. A method of manufacturing an immuno-isolation membrane comprising an inner membrane region, an outer membrane region, and a transition gradient region there between, the method comprising:
depositing an electrospun inner membrane region, wherein the inner membrane region comprises a porous structure with pore sizes of between 0.1 microns to 1.0 micron;
depositing an electrospun outer membrane region, wherein the outer membrane region comprises a porous structure with pore sizes of between 2.0 microns to 50.0 microns; and
wherein the inner membrane region and the outer gradient membrane region are formed with a continuous pore size gradient devoid of lamination or welding between the regions.

7. The method of claim 6, wherein the membrane comprises a polymeric material.

8. The method of claim 7, wherein the polymeric material comprises polytetrafluoroethylene.

9. The method of claim 6, further comprising a step of applying the membrane to an implantable medical device for implantation within the tissue of an animal.

10. The method of claim 6, further comprising a step of depositing a transition region between the inner membrane region and the outer membrane region, and wherein the transition region comprises a porous structure with pores of between approximately 1.0 micron and approximately 10.0 microns.

11. An implantable medical device operable for subcutaneous implantation in an animal, the device comprising:
a hub comprising an internal void;
at least one pod in communication with the hub, the pod comprising an inner cavity operable to receive at least one of cells, a gas and a therapeutic agent, an immuno-isolation member provided adjacent to and exterior to the inner cavity, and a vascularizing membrane provided adjacent to and exterior to the immuno-isolation member;
wherein the hub and the pod are provided in communication with one another by at least one channel extending between the internal void of the hub and the inner cavity of the at least one pod.

12. The implantable medical device of claim 11, wherein the pod further comprises an immune barrier within the vascularizing membrane to prevent ingress of cells from the animal to the inner cavity.

13. The implantable medical device of claim 11, wherein the inner cavity of the at least one pod comprises a population of live cells.

14. The implantable medical device of claim 13, wherein the live cells comprise at least one of islet cells, naturally occurring primary cells, cell lines, genetically engineered cells, and stem cell derived cells.

15. The implantable medical device of claim 11, wherein the device comprises at least two pods.

16. The implantable medical device of claim 11, wherein the hub is in fluid communication with a manifold.

17. The implantable medical device of claim 11, wherein the pod comprises a tapered structure and wherein one end of the pod comprises a greater width than a second, opposing end of the pod.

18. The implantable medical device of claim 11, wherein the hub comprises a pump.

19. The implantable medical device of claim 18, wherein the pump comprises an oxygen pump.

20. The implantable medical device of claim 15, wherein the at least two pods comprise adjacent and partially overlapping pods.

21. The implantable medical device of claim 11, wherein at least one of the hub and the pod comprises electronics.

22. The implantable medical device of claim 11, wherein the hub and the pod comprises at least one energy storage and a power supply.

23. The implantable medical device of claim 11, wherein the pod comprises a sensor.

24. The implantable medical device of claim 11, wherein the device is provided in electronic communication with at least one additional device.

25. The implantable medical device of claim 11, wherein the pod comprises two ports and each of the two ports are in communication with the hub.

26. An implantable medical device operable for subcutaneous implantation in an animal, the device comprising:
a hub comprising an internal void;
at least one pod in communication with the hub, the pod comprising an inner cavity operable to receive at least one of cells, a gas and a therapeutic agent, an immuno-isolation member provided adjacent to and exterior to the inner cavity, and a vascularizing membrane provided adjacent to and exterior to the immuno-isolation member;
wherein the vascularizing membrane comprises an inner region and an outer region, the inner region and the outer region each comprise pores, and a pore-size gradient is provided from the inner region to the outer region;
wherein the inner region comprises a pore size of between about 0.1 microns and about 2.0 micron, and the outer region comprises a pore size of between about 2.0 microns and about 20 microns; and
wherein the hub and the pod are provided in communication with one another by at least one channel extending between the internal void of the hub and the inner cavity of the at least one pod.
